(19) United States
(12) Patent Application Publication
(10) Pub. No.: US 2021/0229895 A1
(43) Pub. Date: Jul. 29, 2021

(54) APPLICATOR

(71) Applicant: GEMÜ GMBH, Emmen (CH)

(72) Inventors: Raimund BISLIN, Büron (CH); Daniel STRASSER, Gossau (CH)

(73) Assignee: GEMÜ GMBH, Emmen (CH)

(21) Appl. No.: 16/972,310

(22) PCT Filed: Oct. 10, 2018
(86) PCT No.: PCT/EP2018/077620

§ 371 Date: Dec. 4, 2020

(30) Foreign Application Priority Data
Jun. 4, 2018 (EP) PCT/EP2018/064643

(51) Int. Cl. B65D 83/00

(52) U.S. Cl. CPC B65D 83/0044

(57) ABSTRACT

An applicator including a cylindrical sleeve and a plunger, which both extend along a common longitudinal axis, wherein the sleeve includes a first end side and a second end side, which is located opposite the first end side, wherein the second end side has formed on it, on the inner side of the sleeve, a first cylindrical shoulder, on which the sleeve can be arranged on a cylindrical container, wherein the first shoulder is followed by a second cylindrical shoulder, in which a complementary cylindrical piston of the plunger is arranged in a displaceable manner, wherein a channel is provided, and extends through the entire plunger along the longitudinal axis, wherein the first end side has formed on it, on the inner side of the sleeve, a third cylindrical shoulder, in which a complementary cylindrical shank of the plunger is arranged in a displaceable manner.

### TECHNICAL FIELD

The present invention relates to an applicator, to a set comprising an applicator and a vial, and to a method for using such a set.

### PRIOR ART

The prior art discloses cylindrical containers that can be sealingly closed, also known as vials, which can be used together awith an applicator in order to apply a liquid, paste or gas that is contained in the vial.

DE 200 22 899 discloses a cylindrical container having a straight bottom, which can be filled with a liquid and which can be sealingly closed at the end side by a membrane. The container can be guided along a longitudinal axis in a guide cylinder of an applicator. A piston is arranged inside the guide cylinder and concentric to the latter. The piston comprises a tip at its end facing towards the container, by which the membrane can be pierced. A continuous channel leads from the tip of the piston, along the longitudinal axis, to an outer side of the applicator. When the container and the applicator are pushed together, the piston tip thus pierces the membrane. By pushing together further, the liquid contained in the container is pressed through the channel to the outer side of the applicator. Due to the straight container bottom and the pointed shape of the piston, the residual amount of liquid remaining in the container when the container and the applicator are fully pushed together is relatively large.

WO 2016/083451 discloses a container having a bottom that tapers to a point, the geometry of which substantially corresponds to that of the piston tip. The residual liquid that remains can thus be reduced. With such a design, most of the residual liquid, that is to say the liquid that cannot be applied, is contained in the channel of the piston.

To keep the channel volume as small as possible, the channels are designed with the smallest possible diameter. Small or thin channels are very difficult or impossible to produce in the injection moulding process or have to be produced afterwards using very fine drills, which greatly increases the manufacturing costs.

### DESCRIPTION OF THE INVENTION

One object of the present invention is to provide an applicator which avoids the abovementioned disadvantages, which is easy to manufacture, and which functions reliably and safely.

This object is achieved by an applicator having the features of claim 1. Further embodiments of the applicator, of a set comprising an applicator and a vial, and of a method for using such a set are defined by the features of further claims.

An applicator according to the invention comprises a cylindrical sleeve and a plunger, both of which extend along a common longitudinal axis. The sleeve comprises a first end side and a second end side, which is located opposite the first end side. At the second end side, a cylindrical first section is formed on the inner side of the sleeve, by means of which the sleeve can be arranged on a cylindrical container. The first section is followed by a cylindrical second section, in which a complementary cylindrical piston of the plunger is arranged in a displaceable manner. A channel is provided, which extends through the entire plunger along the longitudinal axis. At the first end side, a cylindrical third section is formed on the inner side of the sleeve, in which a complementary cylindrical shaft of the plunger is arranged in a displaceable manner. Such a design has the advantage that the piston can be sealingly guided in the sleeve. In addition, such an applicator can be used with many vials while requiring only minor adjustments. With only two components, this construction is simple and reliable. By way of example, the two components are made of plastic. Such an applicator can be produced in an injection moulding process, in particular in an assembly injection moulding process, in which the two parts are produced jointly and simultaneously in the assembled state in a single injection moulding operation. The sleeve and the plunger may be made of the same material or of different materials. The sleeve and the plunger may have a different hardness. By way of example, the sleeve may comprise a hard plastic and the plunger a soft plastic. In such a case, the individual parts can be jointly produced by multi-component assembly injection moulding.

In one embodiment, the diameter of the first section is larger than the diameter of the second section, and the diameter of the second section is larger than the diameter of the third section. The cascade-like gradation of the sections enables easy manufacture of the sleeve and of the plunger.

In one embodiment, a circumferential lateral surface of the first section comprises a first closure element, by which the sleeve can be fastened to the container. Such a closure element may comprise a catch, a bayonet fitting, a threaded portion or a thread. Alternatively, the diameter of the circumferential lateral surface of the section of the sleeve may be somewhat smaller than the outer diameter of the container, as a result of which the sleeve can be clamped onto the container.

In one embodiment, a seal is provided on a circumferential first end face of the first section. The seal may be jointly formed in one piece with the sleeve. By way of example, the seal may be produced together with the sleeve by multi-component injection moulding. Alternatively, a sealing ring or a gasket may be arranged in the first section.

In one embodiment, a chamfer is provided on the second end side, which chamfer adjoins the first section. The chamfer makes it easier to insert the plunger into the sleeve. In addition, the chamfer may be part of a latching recess, as will be described below. A chamfer may likewise be provided on the lateral surface of the second section, which chamfer adjoins the first section. This chamfer likewise makes it easier to insert the plunger into the sleeve at the time of assembly.

In one embodiment, at least one catch is provided at the first end side, which catch projects inwards beyond the lateral surface of the third section and can be releasably latched into at least one groove that is formed in the shaft of the plunger. The latching serves for securing purposes during transportation. In addition, it is advantageous if a force threshold has to be exceeded before the film of the vial can be pierced, and piercing therefore takes place suddenly. The force that is required in order to overcome the latching can be set by the thickness and the thickness profile of the catch.

In one embodiment, the at least one catch is a tab that extends at an angle away from the third section towards the longitudinal axis. By way of example, it extends diagonally inwards. The force that is required in order to overcome the latching can be set by the width of the tabs and the number thereof.

In one embodiment, two or more catches are provided, which are arranged regularly or irregularly distributed around the circumference of the first end side.

At least one rib, which extends radially outwards, may be provided on the outer circumference of the sleeve in the region of the third section. This construction is stable, pleasant to the touch, and enables advantageous manufacture by injection moulding. The at least one rib may be in alignment with the first end side and with the outer circumference of the region of the second section. Two or more ribs may be provided, which are arranged regularly or irregularly distributed around the circumference of the first end side. The ribs may be jointly formed in one piece with the rest of the sleeve.

In one embodiment, the shaft and the piston are jointly formed in one piece with one another, wherein the piston is arranged adjoining an end region of the shaft. The piston comprises a circumferential lateral surface and a bottom, wherein the bottom is arranged on the side of the piston opposite the shaft.

In one embodiment, the piston's lateral surface comprises, on the edge thereof facing towards the shaft, a sealing lug that runs all the way round and that extends outwards beyond the piston's lateral surface. The sealing lug may also be arranged on the edge of the piston's lateral surface that faces away from the shaft. The sealing lug may also be arranged between the two edges on the piston's lateral surface. The piston's lateral surface may also comprise a plurality of sealing lugs, which are arranged spaced apart from one another with respect to the longitudinal axis.

In one embodiment, the bottom of the piston is designed to taper conically inwards, and the channel opens at the centre of the bottom. The angle enclosed in cross-section by the piston bottom and a plane perpendicular to the longitudinal axis may be smaller than 45 degrees, 45 degrees, or larger than 45 degrees. By way of example, the angle may be 30 degrees to 80 degrees. By way of example, the angle may be 40 degrees to 60 degrees. Alternatively, the piston bottom may also be flat.

In one embodiment, at least one piercing element is provided at the edge of the piston-side opening of the channel, which piercing element projects from the bottom of the piston. The opening of the vial can be improved by the piercing element in that the force that is required in order to pierce the film is reduced, and in that the piercing takes place in such a way that there is no detachment of parts of the film that can enter the channel and either clog the channel or are discharged together with the medium to be applied. By way of example, the at least one piercing element is tooth-shaped.

In one embodiment, two or more piercing elements are provided, which are regularly or irregularly arranged at the edge of the piston-side opening of the channel. By way of example, a plurality of mutually adjoining teeth may be provided. The teeth may also be arranged spaced apart from one another. By way of example, three piercing elements are provided, which are each arranged at the edge in a manner offset from one another by 90 degrees. Since no piercing element is provided on one side of the channel edge, the film there is not pierced. The film is therefore folded open around this region, which prevents the film from entering the channel.

In one embodiment, venting grooves are provided between the piercing elements, which venting grooves extend radially outwards from the channel towards the piston skirt over part of the piston bottom. The residual air located in the vial can be guided to the channel by the venting grooves.

In one embodiment, a relief groove is provided in the piston, which relief groove, starting from an end side of the piston close to the shaft, extends annularly along the longitudinal axis in the region of the piston's lateral surface. The relief groove creates a brim-shaped piston, as a result of which the rigidity of the piston's lateral surface is reduced and can better adapt to the sleeve or the vial. The thinner the remaining wall thickness of the piston, the more flexible the piston's lateral surface. By way of example, the radial thickness of the relief groove may substantially correspond to the remaining wall thickness of the piston in the region of the piston's lateral surface.

In one embodiment, the applicator comprises a grip piece having a shaft with a continuous channel, both of which extend along the longitudinal axis, wherein the grip piece can be detachably fastened by its connector to the complementary connector of the plunger. A connector for the grip piece may be provided at an end of the plunger opposite the piston.

In one embodiment, the grip piece comprises at least one grip, which projects laterally from the shaft and which is configured such that at least the fingertip of a user can be placed thereon. A plurality of grips may also be provided on the grip piece. By way of example, two grips may be provided, which are arranged opposite one another with respect to the longitudinal axis. Instead of a plurality of mutually separate grips, a disc that is closed all the way round may also be provided. The grip(s) may be configured in such a way that multiple fingertips can act thereon simultaneously.

In one embodiment, the applicator comprises an attachment having a shaft with a continuous channel, both of which extend along the longitudinal axis, wherein the attachment can be detachably fastened by its connector to the complementary connector of the grip piece. The attachment can be configured in a manner depending on the intended use. By way of example, it may be short or long. As an alternative or in addition, it may be designed as a spray attachment.

In one embodiment, the connectors comprise sections that form connecting pairs, wherein the male partner of the respective connecting pair has a protrusion with a diameter smaller than that of the corresponding shaft, and wherein the female partner of the respective connecting pair has a recess with a diameter smaller than that of the corresponding shaft. By way of example, the plunger comprises a male connector, wherein the grip piece comprises a complementary female connector at one end and a male connector at its other end, and wherein the attachment comprises a female connector complementary to the latter.

In one embodiment, the sections of the connecting pairs each comprise a catch, which can be releasably latched into a complementary groove. A plurality of spaced-apart catches and/or grooves may be provided.

In one embodiment, the sections of the connecting pairs are straight or conical.

In one embodiment, the conical sections of the connecting pairs comprise a Luer cone or a Luer lock connection.

In one embodiment, the angle of the piston bottom is such that the volume enclosed between the piston bottom and the sleeve when the tip of the bottom is in alignment with the first end side of the sleeve substantially corresponds to the volume of all the channels of the applicator. By virtue of this design, the volume enclosed between the piston bottom and the surface of the medium located in the vial corresponds when the tip of the bottom is in alignment with the medium surface. Accordingly, the medium located in the channels can be conveyed substantially in full through this volume by the channels, as a result of which the residual medium to be applied that remains in the applicator is reduced.

The aforementioned embodiments of the applicator can be used in any combination, provided that they do not contradict one another.

A set according to the invention comprises an applicator according to any one of the preceding embodiments and a container, wherein the container is a vial and comprises a bottom and a cylindrical wall adjoining the bottom and running all the way round the latter, which wall extends along the longitudinal axis and the opening of which can be sealingly closed by a film. The applicator can of course also be used together with other containers containing a medium to be applied.

In one embodiment, the outer wall of the container is in alignment with the first section of the sleeve. As a result, the sleeve will be centred and aligned on the container.

In one embodiment, a second closure element is provided on the outer wall of the container and establishes a form-fitting and/or force-fitting connection with the complementary first closure element. The second closure element may be, for example, a thread, a threaded portion or a catch.

In one embodiment, the inner wall of the container is in alignment with the second section of the sleeve. A substantially stepless transition between the sleeve and the container can thus be achieved, as a result of which the plunger can be guided equally well in the sleeve as in the container.

In one embodiment, a chamfer is formed on the inner side of the container opening. This chamfer makes it easier for the plunger to be inserted in the container.

In one embodiment, the chamfer of the container, alone or together with the chamfer of the sleeve, forms a latching recess. The dimensions of the chamfer of the container in the region of where the container makes contact with the sleeve substantially correspond to the dimensions of the chamfer of the sleeve. The sealing lug of the plunger can be releasably latched into the latching recess. When the sealing lug of the plunger is latched into the latching recess, the piston of the plunger is substantially fully inserted into the container and the tip of the piston bottom is at least partially immersed in the medium to be applied. The user has to overcome a predefined force in order to be able to drive the plunger further into the container. As soon as the applied force is greater than that required in order to overcome the latching, the medium to be applied is suddenly conveyed through the channels and is accordingly applied.

The aforementioned embodiments of the set can be used in any combination, provided that they do not contradict one another.

A method according to the invention for using a set according to any one of the above embodiments comprises the steps:
providing a container according to any one of the above embodiments;
providing an applicator according to any one of the above embodiments;
placing the applicator over the opening of the container, which is closed by a film;
displacing the plunger along the longitudinal axis towards the bottom of the container;
piercing the film.

In one embodiment, the method comprises the step:
attaching a grip piece to the connector of the plunger.

In one embodiment, the method comprises the step:
attaching an attachment to the connector of the grip piece.

In one embodiment, the method comprises the steps:
sealing with the sealing lug of the plunger against the second section of the sleeve;
sealing with the sealing lug of the plunger against an inner face of the wall of the container.

In one embodiment, the method comprises the step:
releasably latching the sealing lug of the plunger into the latching recess of the sleeve and/or of the container.

The aforementioned embodiments of the method can be used in any combination, provided that they do not contradict one another.

### BRIEF DESCRIPTION OF THE FIGURES

Exemplary embodiments of the present invention will be explained in greater detail below with reference to figures. Said figures serve only for explanation purposes and are not to be interpreted as limiting. In the figures:

FIG. 1 shows a perspective illustration of an applicator according to the invention, which is placed on a container;

FIG. 2 shows a sectional view through the arrangement of FIG. 1;

FIG. 3 shows a perspective illustration of the plunger of FIG. 1;

FIG. 4 shows a bottom view of the plunger of FIG. 3; and

FIG. 5 shows a sectional view through the plunger of FIG. 4.

### DETAILED DESCRIPTION OF THE INVENTION

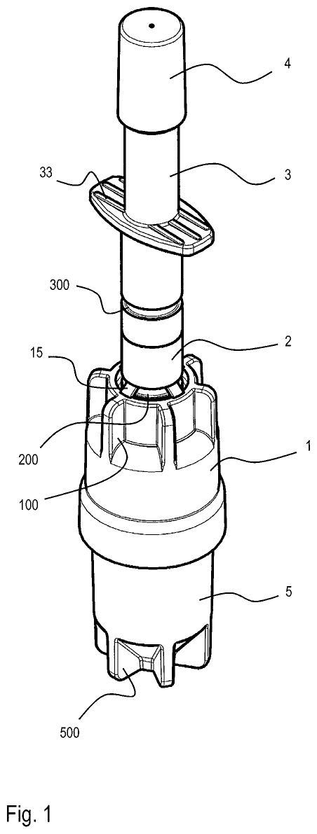

FIG. 1 shows a perspective illustration of an applicator according to the invention, which is placed on a container 5. The applicator comprises a sleeve 1, a plunger 2, a grip piece 3 and an attachment 4.

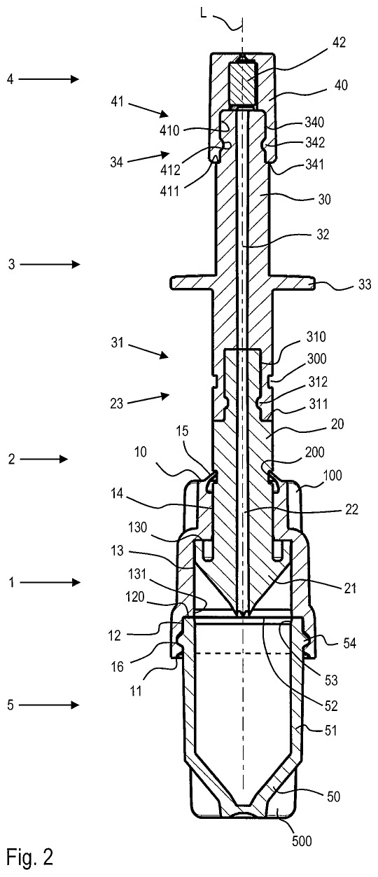

FIG. 2 shows a sectional view through the arrangement of FIG. 1. The sleeve 1 extends along a longitudinal axis L and comprises a first end side 10 and a second end side 11, which is located opposite the first end side. A first section 12 extends on the inner side of the sleeve 1 from the second end side 11 towards the first end side 10. The first section 12 is followed by a second section 13, the diameter of which is smaller than that of the first section 12, as a result of which an annularly extending first end face 120 is formed on the first section 12. A first closure element 16 in the form of a thread is provided in the first section 12. A chamfer 131 is formed on the edge of the second section 13 that faces towards the first section 12. In the embodiment shown, the chamfer has a width of 0.7 millimetres and has an angle of 37 degrees. The second section 13 is followed by a third section 14, the diameter of which is smaller than that of the second section 13, as a result of which an annularly extending second end face 130 is formed on the second section 13. Catches 15 are provided at the first end side 10 of the sleeve 1, which catches, starting from an inner edge of the first end side 10, extend diagonally inwards away from the second end side 11 and towards the longitudinal axis L. In the embodiment shown, four catches 15 are provided, which are regularly distributed around the inner circumference of the second end side 11. The illustrated catches 15 have a width of 1 millimetre and a length of 1 millimetre. The wall thickness of the sleeve 1 is thinnest in the region of the first section 12, somewhat thicker in the region of the third section 14, and thickest in the region of the second section 13. The outer contour of the sleeve is ergonomically designed, tapering by 1 degree in the upward direction. In the embodiment shown, this results in a wall thickness of 0.5 millimetre in the region of the first section, which has a length of 1.8 millimetres, a wall thickness of 1.3 millimetres in the region of the second section, which has a length of 6 millimetres, and a wall thickness of 1.2 millimetres in the region of the third section, which has a length of 3.5 millimetres. In the region of the third section 14, ribs 100 are provided on the outer side of the sleeve 1, said ribs being in alignment with the outer face of the sleeve 1 in the region of the second section 13. In the embodiment shown, six ribs 100 are provided, which are regularly distributed around the circumference. A plunger 2 is arranged in the second section 13 and in the third section 14 of the sleeve 1 in such a way as to be displaceable along the longitudinal axis L.

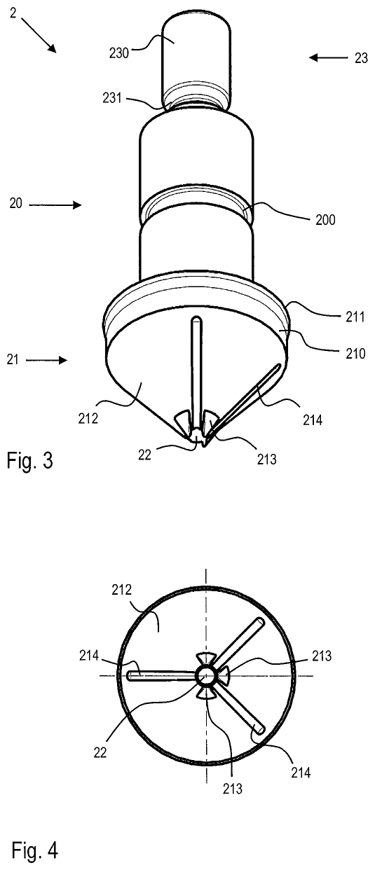

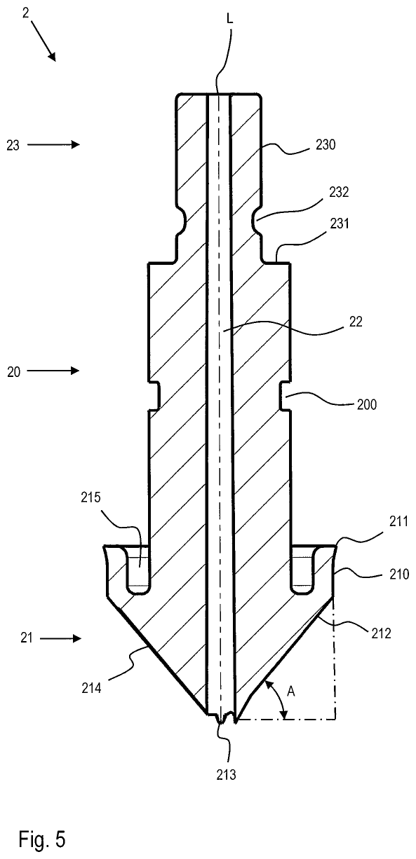

FIG. 3 shows a perspective illustration of the plunger 2 of FIG. 1, FIG. 4 shows a bottom view of the plunger 2 of FIG. 3, and FIG. 5 shows a sectional view through the plunger 2 of FIG. 4. The plunger 2 comprises a shaft 20, which extends in a cylindrical manner along the longitudinal axis L. In the embodiment shown, the shaft has a length of 12 millimetres and a diameter of 5 millimetres. The diameter of the shaft 20 of the plunger 2 corresponds to the diameter of the third section 14 of the sleeve 1, resulting in reliable guidance of the plunger 2 in the sleeve 1 in the assembled state. A piston 21 is formed on a first side of the shaft 20, and a connector 23 is formed on a second side of the shaft 20, which is located opposite the first side. The piston 21 comprises a cylindrical piston's lateral surface 210 with a sealing lug 211, which is formed on the edge of the piston lateral surface 210 that faces towards the connector 23. The diameter of the piston lateral surface 210 corresponds to the diameter of the second section 13 of the sleeve 1, resulting in reliable guidance of the piston 21 in the sleeve 1 in the assembled state. The sealing lug 211 extends all the way round the circumference of the piston lateral surface 210 and projects radially outwards from and beyond the latter. In the assembled state, the sealing lug 211 is compressed by the second section 13, thereby increasing the sealing effect.

A piston bottom 212 is formed on the side of the piston 21 that faces away from the connector 23, which piston bottom tapers conically along the longitudinal axis L. In the embodiment shown, the angle A enclosed by the contour of the piston bottom 212 in cross-section and a plane perpendicular to the longitudinal axis L measures 50 degrees. The plunger 2 comprises a continuous channel 22, which extends from the tip of the piston bottom 212 through the shaft 20 to the connector 23 and through the latter. Three piercing elements 213 are provided at the exit edge of the channel 22 at the piston bottom 212, which piercing elements are arranged offset from one another by an angle of 90 degrees around the circumference of the exit edge. In other words, in each case one piercing element 213 in the form of a piercing tooth is arranged in three out of four quadrants of the piston bottom 212. A venting groove 214 is arranged in each case in the middle between two adjacent teeth 213. Each venting groove 214 extends from the exit edge of the channel 22 substantially radially outwards towards the piston's lateral surface 210 of the piston 21 over part of the piston bottom 212. The lateral end of each venting groove 214 is spaced apart from the edge where the piston bottom 212 meets the piston's lateral surface 210. A compensating groove 215 is formed in the piston 21 of the plunger 2, which compensating groove extends annularly in the extension of the shaft 20, that is to say the inner side of the groove 215 is formed by the shaft 20 and the outer side is formed by an annular surface concentric to the shaft 20. In the embodiment shown, this results in a brim-shaped piston skirt having a wall thickness of 1.7 millimetres. The piston skirt has a diameter of 8 millimetres and a height of 1.8 millimetres. A groove 200 that runs all the way round is formed in the shaft 20 at a distance from the piston 21, into which groove the catches 15 of the sleeve 1 can be latched. The distance between the piston 21 and the groove 200 corresponds to the distance between the second end face 130 of the second section 13 of the sleeve 1 and the catches 15 thereof. The connector 23 of the plunger 2 comprises a section 230, in which a groove 232 that runs all the way round is formed. The diameter of the section 230 is smaller than that of the shaft 20, resulting in an annular side face 231 that runs all the way round. The groove 232 of the connector 23 is formed at a distance from the end side 231. A grip piece 3 having a corresponding connector 31 can be arranged on the connector 23 of the plunger 2. The grip piece 3 comprises a shaft 30, which extends along the longitudinal axis L, wherein a connector 31 for the plunger 2 is provided at one end of said shaft and a connector 34 for an attachment 4 is provided at the other end thereof. A continuous channel 32 extends along the longitudinal axis L through the entire grip piece 3. The connector 31 for the plunger comprises a section 310, in which a catch 312 that runs all the way round is formed. The section 310 is designed as a central bore, the diameter of which corresponds to that of the connector 23 of the plunger 2. The remaining part of the grip piece 3 around the section 310 creates an end side 311 that runs all the way round, which in the assembled state can butt against the corresponding end side 231 of the plunger 2. The circumferential catch 312 of the grip piece 3 is arranged at a distance from the end side 311 such that, in the assembled state, it can latch into the corresponding groove 232 of the plunger 2. A groove 300 that runs all the way round is formed on the outer side of the shaft 30 of the grip piece 3 at a distance from the end side 311. The groove shown has a width of 1 millimetre and a depth of 0.3 millimetre. However, the depth may be up to 0.6 millimetre. The distance between the groove 200 of the plunger 2 and the groove 300 of the grip piece 3 substantially corresponds to the distance between the front edge of the piston skirt of the plunger 2 and the surface of the medium to be applied in the container 5 in the assembled state. Grips 33 are arranged on the shaft 30 at a distance from the groove 300 of the grip piece 3. The grips 33 project laterally from the shaft 30 in such a way that in each case at least one fingertip can be placed thereon. A connector 34 for an attachment 4 is provided on the side of the grip piece 3 that faces away from the plunger 2. The connector 34 comprises a section 340 and a groove 342 formed therein that runs all the way round. The diameter of the section 340 is smaller than the diameter of the shaft 30 of the grip piece 3, resulting in a pin-like connector 34 having an annular end side 341. The groove 342 is formed at a distance from the end side 341. In the assembled state, the attachment 4, which has a complementary connector 41, is arranged on the connector 34 of the grip piece 3. The attachment 4 comprises a shaft 40, which extends along the longitudinal axis L and in the interior of which a channel 42 extends along the entire length thereof. The connector 41 of the attachment 4 comprises a section 410, on which a catch 412 that runs all the way round is formed, which catch engages in the corresponding groove 342 of the grip piece 3 in the assembled state. The section 410 is part of a central bore in the shaft 40. The remaining surface around the bore forms an annular end side 411, which in the assembled state can butt against the corresponding end side 341 of the grip piece 3. In the embodiment shown, the attachment 4 is a spray nozzle attachment, by which the medium to be applied can be atomized. In the assembled state, a container 5 is received in the first section 12 of the sleeve 1. In the embodiment shown, the container 5 is a vial having a bottom 50 and a wall 51 adjoining the bottom 50 and running all the way round the latter. The opening of the vial 5 is sealingly closed by a film 52. A chamfer 53 is formed on the inner edge of the opening. A second closure element 54 is arranged on the outer side of the wall 51 at a distance from the opening; A thread is shown. The outer diameter of the wall 51 corresponds to the inner diameter of the first section 12 of the sleeve 1, and the inner diameter of the wall 51 corresponds to the inner diameter of the second section 13 of the sleeve 1. The bottom 50 is conical, the conicity of the bottom 50 corresponding to that of the piston 21 of the plunger 2. Ribs 500 are arranged on the bottom 50, which ribs extend in the direction of the longitudinal axis L from the conical part of the bottom 50 to the tip of the bottom and which extend laterally as far as the outer side of the wall 51. In the embodiment shown, six ribs 500 are provided, which are arranged regularly distributed around the circumference of the container 5.

LIST OF REFERENCE SIGNS

| | |
|---|---|
| 1 | sleeve |
| 10 | first end side |
| 100 | rib |
| 11 | second end side |
| 12 | first section |
| 120 | first end face |
| 13 | second section |
| 130 | second end face |
| 131 | chamfer |
| 14 | third section |
| 15 | catch |
| 16 | first closure element |
| 2 | plunger |
| 20 | shaft |
| 200 | groove |
| 21 | piston |
| 210 | piston's lateral surface |
| 211 | sealing lug |
| 212 | piston bottom |
| 213 | piercing element |
| 214 | venting groove |
| 215 | compensating groove |
| 22 | channel |
| 23 | connector |
| 230 | section |
| 231 | end side |
| 232 | groove |
| 3 | grip piece |
| 30 | shaft |
| 300 | groove |
| 31 | connector |
| 310 | section |
| 311 | end side |
| 312 | catch |
| 32 | channel |
| 33 | grip |
| 34 | connector |
| 340 | section |
| 341 | end side |
| 342 | catch |
| 4 | attachment |
| 40 | shaft |
| 41 | connector |
| 410 | section |
| 411 | end side |
| 412 | catch |
| 42 | channel |
| 5 | container |
| 50 | bottom |
| 500 | rib |
| 51 | wall |
| 52 | film |
| 53 | chamfer |
| 54 | second closure element |
| L | longitudinal axis |
| A | angle |

1. An applicator comprising a cylindrical sleeve and a plunger, both of which extend along a common longitudinal axis, wherein the sleeve comprises a first end side and a second end side, which is located opposite the first end side, wherein, at the second end side, a cylindrical first section is formed on the inner side of the sleeve, by which the sleeve can be arranged on a cylindrical container, wherein the first section is followed by a cylindrical second section, in which a complementary cylindrical piston of the plunger is arranged in a displaceable manner, wherein a channel is provided, which extends through the entire plunger along the longitudinal axis, wherein at the first end side, a cylindrical third section is formed on the inner side of the sleeve, in which a complementary cylindrical shaft of the plunger is arranged in a displaceable manner.

2. The applicator according to claim 1, wherein the diameter of the first section is larger than the diameter of the second section, and wherein the diameter of the second section is larger than the diameter of the third section.

3. The applicator according to claim 1, wherein a circumferential lateral surface of the first section comprises a first closure element, by which the sleeve can be fastened to the container.

4. The applicator according to claim 1, wherein a seal is provided on a circumferential first end face of the first section.

5. (canceled)

6. The applicator according to claim 1, wherein one, two or more catches are provided at the first end side, which are arranged regularly or irregularly distributed around the circumference of the first end side and which catches project inwards beyond the lateral surface of the third section and can be releasably latched into at least one groove that is formed in the shaft of the plunger.

7. The applicator according to claim 6, wherein the one, two or more catches are tabs that extend at an angle away from the third section towards the longitudinal axis.

8. (canceled)

9. The applicator according to claim 1, wherein the shaft and the piston are jointly formed in one piece with one another, wherein the piston is arranged adjoining an end region of the shaft, wherein the piston comprises a circumferential lateral surface and a bottom, wherein the bottom is arranged on the side of the piston opposite the shaft.

10. The applicator according to claim 9, wherein the piston's lateral surface comprises, on the edge thereof facing towards the shaft, a sealing lug that runs all the way round and that extends outwards beyond the piston's lateral surface.

11. The applicator according to claim 1, wherein the bottom of the piston is designed to taper conically inwards, and wherein the channel opens at the centre of bottom.

12. The applicator according to claim 1, wherein one, two or more piercing elements are provided at the edge of the piston-side opening of the channel, which are regularly or irregularly arranged at the edge of the piston-side opening of the channel and which piercing elements project from the bottom of the piston.

13. (canceled)

14. The applicator according to claim 1, wherein venting grooves are provided between the piercing elements, which venting grooves extend radially outwards from the channel towards the piston skirt over part of the piston bottom.

15. The applicator according to claim 1, wherein a relief groove is provided in the piston, which relief groove, starting from an end side of the piston close to the shaft, extends annularly along the longitudinal axis in the region of the piston's lateral surface.

16. The applicator according to claim 1, comprising a grip piece having a shaft with a continuous channel, both of which extend along the longitudinal axis, wherein the grip piece can be detachably fastened by its connector to the complementary connector of the plunger.

17. The applicator according to claim 16, wherein the grip piece comprises at least one grip, which projects laterally from the shaft and which is configured such that at least the fingertip of a user can be placed thereon.

18. The applicator according to claim 1, comprising an attachment having a shaft with a continuous channel, both of which extend along the longitudinal axis, wherein the attachment can be detachably fastened by its connector to the complementary connector of the grip piece.

19. The applicator according to claim 18, wherein the connectors comprise sections that form connecting pairs, wherein the male partner of the respective connecting pair has a protrusion with a diameter smaller than that of the corresponding shaft, and wherein the female partner of the respective connecting pair has a recess with a diameter smaller than that of the corresponding shaft.

20. The applicator according to claim 19, wherein the sections of the connecting pairs each comprise a catch, which can be releasably latched into a complementary groove.

21. The applicator according to claim 19, wherein the sections of the connecting pairs are straight or conical.

22. The applicator according to claim 19, wherein the conical sections of the connecting pairs comprise a Luer cone or a Luer lock connection.

23. The applicator according to claim 11, wherein the angle of the piston bottom is such that the volume enclosed between the piston bottom and the sleeve when the tip of the bottom is in alignment with the first end face of the sleeve substantially corresponds to the volume of all the channels of the applicator.

24. A set comprising an applicator according to claim 1 and a container, wherein the container is a vial and comprises a bottom and a cylindrical wall adjoining the bottom and running all the way round the latter, which wall extends along the longitudinal axis and the opening of which can be sealingly closed by a film.

25. The set according to claim 24, wherein the outer wall of the container is in alignment with the first section of the sleeve.

26. The set according to claim 24, wherein a second closure element is provided on the outer wall of the container and establishes a form-fitting and/or force-fitting connection with the complementary first closure element.

27. The set according to claim 24, wherein the inner wall of the container is in alignment with the second section of the sleeve.

28. The set according to claim 24, wherein a chamfer is formed on the inner side of the container opening.

29. The set according to claim 28, wherein the chamfer of the container, alone or together with a chamfer of the sleeve, which chamfer adjoins the first section of the sleeve, forms a latching recess, wherein the dimensions of the chamfer of the container in the region where the container makes contact with the sleeve substantially correspond to the dimensions of the chamfer of the sleeve, and wherein the sealing lug of the plunger can be releasably latched into the latching recess.

30-34. (canceled)
